(19) United States
(12) Patent Application Publication
(10) Pub. No.: US 2002/0176884 A1
(43) Pub. Date: Nov. 28, 2002

(54) Process for the fermentative preparation of D-pantothenic acid and/or salts thereof

(75) Inventors: Nicole Dusch, Werther (DE); Achim Marx, Halle (DE); Walter Pfefferle, Halle (DE); Georg Thierbach, Bielefeld (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: Degussa AG, Duesseldorf (DE)

(21) Appl. No.: 09/983,167

(22) Filed: Oct. 23, 2001

(30) Foreign Application Priority Data
Apr. 3, 2001 (DE) 101 16 519.6

(51) Int. Cl. A23K 1/165; C12P 13/04; C12N 1/21

(52) U.S. Cl. 424/442; 435/252.3; 435/106

(57) ABSTRACT

The invention provides a process for the fermentative preparation of D-pantothenic acid and/or salts thereof or feedstuffs additives comprising these by fermentation of coryneform bacteria, in particular those which already produce D-pantothenic acid, wherein the nucleotide sequence(s) in the coryneform bacteria which code(s) for the glyA gene is (are) enhanced, in particular over-expressed.

### FIELD OF THE INVENTION

This invention relates to a process for the fermentative preparation of D-pantothenic acid and/or salts thereof or mixtures comprising these using coryneform bacteria in which at least the glyA gene is enhanced.

### PRIOR ART

Pantothenic acid is produced worldwide in an order of magnitude of several thousand tons a year. It is used inter alia in human medicine, in the pharmaceuticals industry and in the foodstuffs industry. A large portion of the pantothenic acid produced is used for nutrition of stock animals such as poultry and pigs. Demand is increasing.

Pantothenic acid can be prepared by chemical synthesis, or biotechnologically by fermentation of suitable microorganisms in suitable nutrient solutions. In the chemical synthesis, DL-pantolactone is an important precursor. It is prepared in a multi-stage process from formaldehyde, isobutylaldehyde and cyanide, and in further process steps, the racemic mixture is separated, D-pantolactone is subjected to a condensation reaction with β-alanine, and D-pantothenic acid is obtained in this way.

The typical commercial form is the calcium salt of D-pantothenic acid. The calcium salt of the racemic mixture of D,L-pantothenic acid is also customary.

The advantage of the fermentative preparation by microorganisms lies in the direct formation of the desired stereoisomeric form, that is to say the D-form, which is free from L-pantothenic acid.

Processes for the preparation of D-pantothenic acid with the aid of Corynebacterium glutamicum are known only in some instances in the literature. Sahm and Eggeling (Applied and Environmental Microbiology 65(5), 1973-1979, (1999)) thus report on the influence of over-expression of the panB and panC genes and Dusch et al. (Applied and Environmental Microbiology 65(4), 1530-1539, (1999)) report on the influence of the panD gene on the formation of D-pantothenic acid.

### OBJECT OF THE INVENTION

The inventors had the object of providing new measures for improved fermentative preparation of D-pantothenic acid and/or salts thereof, and animal feedstuffs additives comprising these.

### SUMMARY OF THE INVENTION

When D-pantothenic acid or pantothenic acid or pantothenate are mentioned in the following text, this means not only the free acids but also the salts of D-pantothenic acid, such as e.g. the calcium, sodium, ammonium or potassium salt.

The invention provides a process for the preparation of D-pantothenic acid and/or salts thereof or feedstuffs additives additives comprising these by fermentation of coryneform bacteria, in particular those which already produce D-pantothenic acid, in which

a) the nucleotide sequence(s) in the bacteria which code(s) for the endogenous glyA gene is (are) enhanced, in particular over-expressed, under conditions which are suitable for the production of serine hydroxymethyl transferase,

b) the D-pantothenic acid and/or salts thereof in the medium or in the cells of the microorganisms are concentrated, and

c) after conclusion of the fermentation, the desired products are isolated, the biomass and/or optionally further constituents of the fermentation broth being separated off in an amount of ≧0 to 100%,

D-pantothenic acid being produced by the bacteria.

The invention also provides a process in which, after conclusion of the fermentation, some or all of the biomass remains in the fermentation broth, and the broth obtained in this way is processed, optionally after concentration, to a solid mixture which comprises D-pantothenic acid and/or salts thereof and also comprises further constituents of the fermentation broth.

### DETAILED DESCRIPTION OF THE INVENTION

The term “enhancement” in this connection describes the increase in the intracellular activity of one or more enzymes or proteins in a microorganism which are coded by the corresponding DNA, for example by increasing the number of copies of the gene or genes, using a potent promoter or a gene or allele which codes for a corresponding enzyme or protein with a high activity, and optionally combining these measures.

By enhancement measures, in particular over-expression, the activity or concentration of the corresponding protein is in general increased by at least 10%, 25%, 50%, 75%, 100%, 150%, 200%, 300%, 400% or 500%, up to a maximum of 1000% or 2000%, based on that of the wild-type protein or the activity or concentration of the protein in the starting microorganism.

The microorganisms which the present invention provides can produce D-pantothenic acid from glucose, sucrose, lactose, fructose, maltose, molasses, starch, cellulose or from glycerol and ethanol. They are representatives of coryneform bacteria, in particular of the genus Corynebacterium. Of the genus Corynebacterium, there may be mentioned in particular the species Corynebacterium glutamicum (C. glutamicum), which is known among experts for its ability to produce L-amino acids.

Suitable strains of the genus Corynebacterium, in particular of the species Corynebacterium glutamicum, are, for example, the known wild-type strains

Corynebacterium glutamicum ATCC13032

Corynebacterium acetoglutamicum ATCC15806

Corynebacterium acetoacidophilum ATCC13870

Corynebacterium thermoaminogenes FERM BP-1539

Brevibacterium flavum ATCC14067

Brevibacterium lactofermentum ATCC13869 and

Brevibacterium divaricatum ATCC14020

and D-pantothenic acid-producing mutants prepared therefrom, such as, for example

Corynebacterium glutamicum ATCC13032ΔilvA/pEC7panBC

Cornyebacterium glutamicum ATCC13032/pND-D2

It has been found that coryneform bacteria produce pantothenic acid in an improved manner after over-expression of the glyA gene which codes for serine hydroxymethyl transferase.

The glyA gene codes for the enzyme serine hydroxymethyl transferase (EC 2.1.2.1). The nucleotide sequence of the glyA gene has been described in Japanese Laid-Open Specification JP-A-08107788. The glyA gene described in the text reference mentioned can be used according to the invention. Alleles of the glyA gene which result from the degeneracy of the genetic code or due to sense mutations of neutral function and lead to no fundamental change in the activity of the protein can furthermore be used. Such mutations are also called, inter alia, neutral substitutions.

To achieve an enhancement (e.g. over-expression), e.g. the number of copies of the corresponding genes is increased, or the promoter and regulation region or the ribosome binding site upstream of the structural gene is mutated. Expression cassettes which are incorporated upstream of the structural gene act in the same way. By inducible promoters, it is additionally possible to increase the expression in the course of fermentative pantothenic acid formation. The expression is likewise improved by measures to prolong the life of the m-RNA. Furthermore, the enzyme activity is also increased by preventing the degradation of the enzyme protein. The genes or gene constructs are either present here in plasmids with a varying number of copies, or are integrated and amplified in the chromosome. Alternatively, an over-expression of the genes in question can furthermore be achieved by changing the composition of the media and the culture procedure.

Instructions in this context can be found by the expert, inter alia, in Martin et al. (Bio/Technology 5, 137-146 (1987)), in Guerrero et al. (Gene 138, 35-41 (1994)), Tsuchiya and Morinaga (Bio/Technology 6, 428-430 (1988)), in Eikmanns et al. (Gene 102, 93-98 (1991)), in EP 0 472 869, in U.S. Pat. No. 4,601,893, in Schwarzer and Pühler (Bio/Technology 9, 84-87 (1991), in Reinscheid et al. (Applied and Environmental Microbiology 60, 126-132 (1994)), in LaBarre et al. (Journal of Bacteriology 175, 1001-1007 (1993)), in Patent Application WO 96/15246, in Malumbres et al. (Gene 134, 15-24 (1993)), in Japanese Laid-Open Specification JP-A-10-229891, in Jensen and Hammer (Biotechnology and Bioengineering 58, 191-195 (1998)) and in known textbooks of genetics and molecular biology.

For example, the glyA gene can be over-expressed with the aid of plasmids.

Suitable plasmids are those which are replicated in coryneform bacteria. Numerous known plasmid vectors, such as e.g. pZ1 (Menkel et al., Applied and Environmental Microbiology (1989), 64: 549-554), pEKEx1 (Eikmanns et al., Gene 102:93-98 (1991)), or pHS2-1 (Sonnen et al., Gene 107:69-74 (1991)) are based on the cryptic plasmids pHM1519, pBL1 or pGA1. Other plasmid vectors, such as e.g. those based on pCG4 (U.S. Pat. No. 4,489,160), or pNG2 (Serwold-Davis et al., FEMS Microbiology Letters 66, 119-124 (1990)), or pAG1 (U.S. Pat. No. 5,158,891), can be used in the same manner.

Plasmid vectors which are moreover suitable are those with the aid of which the process of gene amplification by integration into the chromosome can be used, as has been described, for example, by Reinscheid et al. (Applied and Environmental Microbiology 60, 126-132 (1994)) for duplication or amplification of the hom-thrB operon.

In this method, the complete gene is cloned in a plasmid vector which can replicate in a host (typically E. coli), but not in C. glutamicum. Possible vectors are, for example, pSUP301 (Simon et al., Bio/Technology 1, 784-791 (1983)), pK18mob or pK19mob (Schäfer et al., Gene 145, 69-73 (1994)), pGEM-T (Promega Corporation, Madison, Wis. USA), pCR2.1-TOPO (Shuman (1994). Journal of Biological Chemistry 269:32678-84; U.S. Pat. No. 5,487,993), pCR®Blunt (Invitrogen, Groningen, Holland; Bernard et al., Journal of Molecular Biology, 234: 534-541 (1993)) or pEM1 (Schrumpf et al, 1991, Journal of Bacteriology 173:4510-4516). The plasmid vector which contains the gene to be amplified is then transferred into the desired strain of C. glutamicum by conjugation or transformation. The method of conjugation is described, for example, by Schäfer et al. (Applied and Environmental Microbiology 60, 756-759 (1994)). Methods for transformation are described, for example, by Thierbach et al. (Applied Microbiology and Biotechnology 29, 356-362 (1988)), Dunican and Shivnan (Bio/Technology 7, 1067-1070 (1989)) and Tauch et al. (FEMS Microbiological Letters 123, 343-347 (1994)). After homologous recombination by means of a “cross over” event, the resulting strain contains at least two copies of the gene in question.

For production of pantothenic acid, it may additionally be advantageous to enhance one or more further genes which code for enzymes of the serine biosynthesis pathway, the keto-isovaleric acid biosynthesis pathway, the aspartic acid biosynthesis pathway, the pantothenic acid biosynthesis pathway, glycolysis or anaplerosis, in addition to the endogenous glyA gene.

Thus, in addition one or more of the enodgenous genes chosen from the group consisting of:

the serA gene which codes for 3-phosphoglycerate dehydrogenase or a serA allele which codes for a “feed back” resistant phosphoglycerate dehydrogenase (U.S. Pat. No. 6,037,154),

the serC gene which codes for phosphoserine transaminase, (WO01/00843)

the serB gene which codes for phosphoserine phosphatase, (WO01/00843)

the ilvBN operaon which codes for acetohydroxy-acid synthase (Journal of Bacteriology 175, 5595-5603 (1993)),

the ilvD gene which codes for dihydroxy-acid dehydratase (JP-A-1996089249),

the ilvC gene which codes for acetohydroxy-acid isomeroreductase (EP-A-1001027),

the panB gene which codes for ketopantoate hydroxymethyl transferase (EP-A-1006189),

the panC gene which codes for pantothenate synthetase (EP-A-1006189),

the panD gene which codes for aspartate decarboxylase (EP-A-1006192),

the pfkA gene which codes for phosphofructokinase (DE: 19956133.8 and DE: 10030702.7) and

the pyc gene which codes for pyruvate carboxylase (DE-A-19831609 31 609 and DE: 19943055.1)

can be enhanced, in particular over-expressed.

It may furthermore be advantageous for the production of pantothenic acid, in addition to the enhancement of the glyA gene which codes for serine hydroxymethyl transferase, for one or more of the genes chosen from the group consisting of:

the pck gene which codes for phosphoenol pyruvate carboxykinase (PEP carboxykinase) (DE: 19950409.1) and

the poxB gene which codes for pyruvate oxidase (DE: 19951975.7 and DE: 10048604.5)

to be attenuated, in particular suppressed or for the expression thereof to be reduced.

The term “attenuation” in this connection describes the reduction or elimination of the intracellular activity of one or more enzymes (proteins) in a microorganism which are coded by the corresponding DNA, for example by using a weak promoter or using a gene or allele which codes for a corresponding enzyme (protein) with a low activity or inactivates the corresponding gene or enzyme (protein), and optionally combining these measures.

By attenuation measures, including reduction in expression, the activity or concentration of the corresponding protein is in general reduced to 0 to 75%, 0 to 50%, 0 to 25%, 0 to 10% or 0 to 5% of the activity or concentration of the wild-type protein or of the activity or concentration of the protein in the starting microorganism.

In addition to over-expression of the glyA gene it may furthermore be advantageous for the production of D-pantothenic acid to eliminate undesirable side reactions (Nakayama: “Breeding of Amino Acid Producing Microorganisms”, in: Overproduction of Microbial Products, Krumphanzl, Sikyta, Vanek (eds.), Academic Press, London, UK, 1982). Bacteria in which the metabolic pathways which reduce the formation of D-pantothenic acid are at least partly eliminated can be employed in the process according to the invention.

The microorganisms produced according to the invention can be cultured in the batch process (batch culture), the fed batch (feed process) or the repeated fed batch process (repetitive feed process). A summary of known culture methods is described in the textbook by Chmiel (Bioprozesstechnik 1. Einführung in die Bioverfahrenstechnik (Gustav Fischer Verlag, Stuttgart, 1991)) or in the textbook by Storhas (Bioreaktoren und periphere Einrichtungen (Vieweg Verlag, Braunschweig/Wiesbaden, 1994)).

The culture medium to be used must meet the requirements of the particular strains in a suitable manner. Descriptions of culture media for various microorganisms are contained in the handbook “Manual of Methods for General Bacteriology” of the American Society for Bacteriology (Washington D.C., USA, 1981). Sugars and carbohydrates, such as e.g. glucose, sucrose, lactose, fructose, maltose, molasses, starch and cellulose, oils and fats, such as e.g. soya oil, sunflower oil, groundnut oil and coconut fat, fatty acids, such as e.g. palmitic acid, stearic acid and linoleic acid, alcohols, such as e.g. glycerol and ethanol, and organic acids, such as e.g. acetic acid, can be used as the source of carbon. These substances can be used individually or as a mixture.

Organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture.

Phosphoric acid, potassium dihydrogen phosphate or dipotassium hydrogen phosphate or the corresponding sodium-containing salts can be used as the source of phosphorus. The culture medium must furthermore comprise salts of metals, such as e.g. magnesium sulfate or iron sulfate, which are necessary for growth. Finally, essential growth substances, such as amino acids and vitamins, can be employed in addition to the above-mentioned substances. Precursors of pantothenic acid, such as aspartate, β-alanine, ketoisovalerate, ketopantoic acid or pantoic acid and optionally salts thereof, can moreover be added to the culture medium. The starting substances mentioned can be added to the culture in the form of a single batch, or can be fed in during the culture in a suitable manner.

Basic compounds, such as sodium hydroxide, potassium hydroxide, ammonia or aqueous ammonia, or acid compounds, such as phosphoric acid or sulfuric acid, can be employed in a suitable manner to control the pH of the culture.

For the preparation of alkaline earth metal salts of pantothenic acid, in particular the calcium salt, it is equally possible to add the suspension or solution of an inorganic compound containing an alkaline earth metal, such as, for example, calcium hydroxide, or of an organic compound, such as the alkaline earth metal salt of an organic acid, for example calcium acetate, continuously or discontinuously during the fermentation. In this manner, the cation necessary for preparation of the desired alkaline earth metal salt of D-pantothenic acid is introduced into the fermentation broth directly in the desired amount, in general in a ratio of 0.8 to 1.2:1, based on the pantothenic acid, preferably in stoichiometric amounts.

Antifoams, such as e.g. fatty acid polyglycol esters, can be employed to control the development of foam. Suitable substances having a selective action, e.g. antibiotics, can be added to the medium to maintain the stability of plasmids. To maintain aerobic conditions, oxygen or oxygen-containing gas mixtures, such as e.g. air, are introduced into the culture. The temperature of the culture is usually 25° C. to 45° C., and preferably 30° C. to 40° C. Culturing is continued until a maximum of D-pantothenic acid has formed. This target is usually reached within 10 hours to 160 hours.

The D-pantothenic acid contained in the fermentation broth or the corresponding salts of D-pantothenic acid can then be isolated and purified in accordance with the prior art.

It is also possible for the fermentation broths comprising D-pantothenic acid and/or salts thereof preferably first to be freed from all or some (≧0 to 100%) of the biomass by known separation methods, such as, for example, centrifugation, filtration, decanting or a combination thereof. However, it is also possible to leave the biomass in its entirety in the fermentation broth. In general, the suspension or solution is preferably concentrated and worked up to a powder, for example with the aid of a spray dryer or a freeze-drying unit. This powder in then in general converted by suitable compacting or granulating processes, e.g. also build-up granulation, into a coarser-grained, free-flowing, storable and largely dust-free product with a particle size distribution of 20 to 2000 μm, in particular 100 to 1400 μm. In the granulation or compacting it is advantageous to employ conventional organic or inorganic auxiliary substances or carriers, such as starch, gelatin, cellulose derivatives or similar substances, such as are conventionally used as binders, gelling agents or thickeners in foodstuffs or feedstuffs processing, or further substances, such as, for example, silicas, silicates or stearates.

Alternatively, the fermentation product, with or without further of the conventional constituents of the fermentation broth, can be absorbed on to an organic or inorganic carrier substance which is known and conventional in feedstuffs processing, such as, for example, silicas, silicates, grits, brans, meals, starches, sugars or others, and/or stabilized with conventional thickeners or binders. Use examples and processes in this context are described in the literature (Die Mühle+Mischfuttertechnik 132 (1995) 49, page 817).

D-Pantothenic acid and/or the desired salt of D-pantothenic acid or a formulation comprising these compounds is optionally added at a suitable process stage in order to achieve or establish the desired content of pantothenic acid or the desired salt.

The desired content is in general in the range from 20 to 80 wt. % (dry weight).

The concentration of pantothenic acid can be determined with known chemical (Velisek; Chromatographic Science 60, 515-560 (1992)) or microbiological methods, such as e.g. the Lactobacillus plantarum test (DIFCO MANUAL, 10^{th} Edition, p. 1100-1102; Michigan, USA).

The present invention is explained in more detail in the following with the aid of embodiment examples.

For this purpose, experiments were carried out with the isoleucine-requiring strain ATCC13032ΔilvA, the plasmid pND-D2 and the plasmid pVWEx2glyA.

A pure culture of the strain ATCC13032ΔilvA was deposited on Oct. 21, 1998 as DSM12455 at the Deutsche Sammlung für Mikroorganismen und Zellkulturen [German Collection of Microorganisms and Cell Cultures] in Braunschweig (Germany) in accordance with the Budapest Treaty. The plasmid pND-D2 containing the panD gene is described in Dusch et al. (Applied and Environmental Microbiology 65(4), 1530-1539 (1999)) and was also deposited in the form of a pure culture of the strain Corynebacterium glutamicum ATCC13032/pND-D2 on Oct. 5, 1998 as DSM12438 at the Deutsche Sammlung für Mikroorganismen und Zellkulturen [German Collection of Microorganisms and Cell Cultures] in Braunschweig (Germany) in accordance with the Budapest Treaty. The plasmid pVWEx2glyA is described in EP-A-1106695. It was deposited in the form of a pure culture of the Escherichia coli strain DH5alphamcr/pVWEx2glyA on Aug. 3, 2001 at the Deutsche Sammlung für Mikroorganismen und Zellkulturen [German Collection of Microorganisms and Cell Cultures] in Braunschweig (Germany) in accordance with the Budapest Treaty. A map of the plasmid pVWEx2glyA is shown in FIG. 1.

### EXAMPLE 1

### Preparation of the Strain ATCC13032ΔilvA/pND-D2, pVWEx2glyA

After electroporation (Tauch et.al., 1994, FEMS Microbiological Letters, 123:343-347) of the plasmid pND-D2 in the C. glutamicum strain ATCC13032ΔilvA and subsequent selection on LB agar (Lennox, 1955, Virology, 1:190-206), which had been supplemented with 25 μg/ml kanamycin, the strain ATCC13032ΔilvA/pND-D2 was obtained.

After electroporation of the plasmid pVWEx2glyA in the C. glutamicum strain ATCC13032ΔilvA/pND-D2 and subsequent selection on LB agar, which had been supplemented with 25 Δg/ml kanamycin and 10 Δg/ml tetracycline, the strain ATCC13032ΔilvA/pND-D2, pVWEx2glyA was obtained.

### EXAMPLE 2

### Preparation of Pantothenic Acid

The formation of pantothenate by the C. glutamicum strains ATCC13032ΔilvA/pND-D2 and ATCC13032ΔilvA/pND-D2, pVWEx2glyA was tested in medium CGXII (Keilhauer et al., 1993, Journal of Bacteriology, 175:5595-5603; table 1), which had been supplemented with 25 μg/ml kanamycin, 2 mM isoleucine and in the case of the strain ATCC13032ΔilvA/pND-D2, pVWEx2glyA with additionally 10 μg/ml tetracycline.

This medium is called C. glutamicum test medium in the following. In each case 50 ml of freshly prepared C. glutamicum test medium were inoculated with a 16 hours old preculture of the same medium such that the optical density of the culture suspension (O.D.₅₈₀) at the start of incubation was 0.1. The cultures were incubated at 30° C. and 130 rpm. After incubation for 5 hours, IPTG (isopropyl β-D-thiogalactoside) was added in a final concentration of 1 mM. After incubation for 48 hours the optical density (O.D.₅₈₀) of the culture was determined and the cells were then removed by centrifugation at 5000 g for 10 minutes and the supernatant subjected to sterile filtration.

A Novaspec II photometer from Pharmacia (Freiburg, Germany) was employed at a measurement wavelength of 580 nm for determination of the optical density.

The D-pantothenate in the culture supernatant was quantified by means of Lactobacillus plantarum ATCC 8014 in accordance with the instructions in the handbook of DIFCO (DIFCO MANUAL, 10^{th} Edition, p. 1100-1102; Michigan, USA). The hemi-calcium salt of pantothenate from Sigma (Deisenhofen, Germany) was used for the calibration. The result is shown in table 2.

TABLE 1

| | | |
|---|---|---|
| Substance | Amount per liter | Comments |

| | | | |
|---|---|---|---|
| (NH₄)₂ SO₂ | 20 | g | |
| Urea | 5 | g | |
| KH₂PO₄ | 1 | g | |
| K₂HPO₄ | 1 | g | |
| MgSO₄ * 7 H₂O | 0.25 | g | |
| MOPS | 42 | g | |
| CaCl₂ | 10 | mg | |
| FeSO₄ * 7 H₂O | 10 | mg | |
| MnSO₄ * H₂O | 10 | mg | |
| ZnSO₄ * 7 H₂O | 1 | mg | |
| CuSO₄ | 0.2 | mg | |
| NiCl₂ * 6 H₂O | 0.02 | mg | |
| Biotin | 0.5 | mg | |
| Glucose | 40 | g | autoclave |
| | | | separately |
| Protocatechuic acid | 0.03 | mg | sterile |
| | | | filtration |

TABLE 2

| | Cell density | Concentration |
|---|---|---|
| Strain | OD580 | (ng/ml) |
| ATCC13032ΔilvA/pND-D2 | 14.9 | 30 |
| ATCC13032ΔilvA/pND-D2, | 14.8 | 51 |
| pVWEx2glyA | | |

### BRIEF DESCRIPTION OF THE FIGURE

FIG. 1: Map of the plasmid pVWEx2glyA

The base pair numbers stated are approximate values obtained in the context of reproducibility of measurements.

The abbreviations and designations used have the following meaning.

Tet: Resistance gene for tetracycline

lacI: lacI repressor

Ptac: tac promoter

glyA: Serine hydroxymethyl transferase gene from C. glutamicum

BclI: Cleavage site of the restriction enzyme BamHI

NarI: Cleavage site of the restriction enzyme NarI

SalI: Cleavage site of the restriction enzyme SalI

XbaI: Cleavage site of the restriction enzyme XbaI

What is claimed is:

1. Process for the preparation of D-pantothenic acid and/or salts thereof or feedstuffs additives comprising these by fermentation of coryneform bacteria, in particular those which already produce D-pantothenic acid, in which
a) the nucleotide sequence(s) in the bacteria which code(s) for the endogenous glyA gene is (are) enhanced, in particular over-expressed, under conditions which are suitable for the production of serine hydroxymethyl transferase,
b) the D-pantothenic acid and/or salts thereof in the medium or in the cells of the microorganisms are concentrated, and
c) after conclusion of the fermentation, the desired products are isolated, the biomass and/or optionally further constituents of the fermentation broth being separated off in an amount of ≧0 to 100%,
D-pantothenic acid being produced by the microorganisms.

2. Process according to claim 1, wherein the fermentation is carried out in the presence of alkaline earth metal salts, these being added continuously or discontinuously in the desired amount, and a product comprising alkaline earth metal salts of D-pantothenic acid or consisting of these being obtained.

3. Process according to claim 1, wherein coryneform bacteria of the genus Corynebacterium are employed.

4. Process according to claim 3, wherein the coryneform bacteria of the genus Corynebacterium originate from the species Corynebacterium glutamicum.

5. Process according to claim 1, wherein, in addition to the endogenous glyA gene, one or more of the endogenous genes chosen from the groups consisting of:
5.1 the serA gene which codes for 3-phosphoglycerate dehydrogenase or a serA allele which codes for a “feed back” resistant phosphoglycerate dehydrogenase,
5.2 the serC gene which codes for phosphoserine transaminase,
5.3 the serb gene which codes for phosphoserine phosphatase,
5.4 the ilvBN operon which codes for acetohydroxy-acid synthase,
5.5 the ilvD gene which codes for dihydroxy-acid dehydratase,
5.6 the ilvC gene which codes for acetohydroxy acid isomeroreductase,
5.7 the panB gene which codes for ketopantoate hydroxymethyl transferase,
5.8 the panC gene which codes for pantothenate synthetase,
5.9 the panD gene which codes for aspartate decarboxylase,
5.10 the panC gene which codes for pantothenate synthetase,
5.11 the pfkA gene which codes for phosphofructokinase and
5.12 the pyc gene which codes for pyruvate carboxylase,
is or are enhanced, in particular over-expressed.

6. Process according to claim 1, wherein bacteria are used in which the metabolic pathways which reduce the formation of D-pantothenic acid are at least partially suppressed, in particular the pck gene which codes for phosphoenol pyruvate carboxykinase (PEP carboxykinase) and/or the poxB gene which codes for pyruvate oxidase, are suppressed or expressed at a low level.

7. Process according to claim 1, wherein the activity or concentration of the glyA gene product (protein) is increased by 10 to 2000%, based on the wild-type protein or the protein in the starting microorganism.

8. Process according to claim 1, wherein transformed strains in which the glyA gene is integrated in plasmids or in the chromosome and enhanced are employed.

9. A vector for expression of the glyA gene of C. glutamicum, which comprises a promoter and the gene sequence.

10. Coryneform bacteria, in particular of the genus Corynebacterium, transformed with a vector according to claim 9.

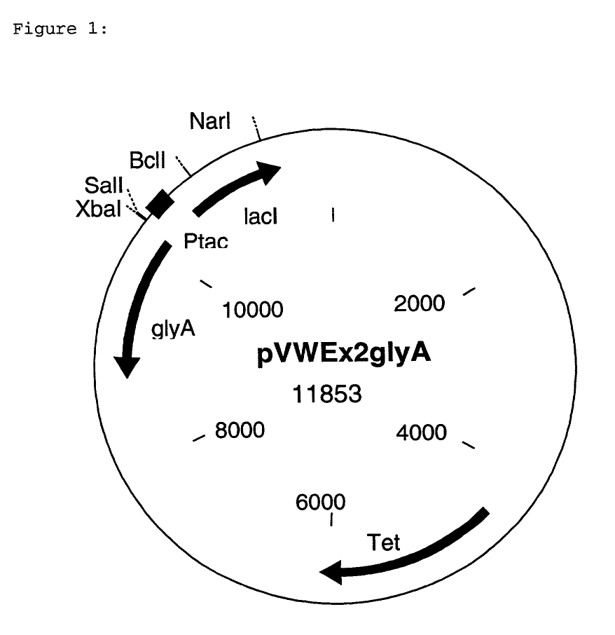

11. Coryneform bacteria, in particular of the genus Corynebacterium, transformed with the plasmid pVWEx2glyA, shown in FIG. 1.

12. Process for the preparation of D-pantothenic acid and/or salts thereof by fermentation of microorganisms according to claims 10 or 11.

13. Process according to claim 1, in which
a) from a fermentation broth obtained by fermentation and comprising D-pantothenic acid and/or salts thereof, the biomass and/or optionally the constituents of the fermentation broth are separated off in an amount of ≧0 to 100%,
b) the mixture obtained in this way is optionally concentrated, and
c) the mixture comprising the pantothenic acid and/or the pantothenate is converted into a free-flowing form by suitable measures, and
d) a free-flowing animal feedstuffs additive with a particle size distribution of 20 to 2000 μm, in particular 100 to 1400 μm, is prepared therefrom by suitable measures.

14. Process for the preparation of animal feedstuffs additives according to claim 2 with a content of D-pantothenic acid and/or salts thereof, chosen from the group consisting of the magnesium or calcium salt, in which
a) water is optionally removed from the fermentation broth (concentration),
b) the biomass formed during the fermentation is separated off in an amount of ≧0 to 100%,
c) optionally one or more of the compounds mentioned are added to the fermentation broths treated according to a) and b), the amount of compounds added being such that the total concentration thereof in the animal feedstuffs additive is in the range from about 20 to 80 wt. % (dry weight), and
d) the animal feedstuffs additive is prepared in the desired powder or, preferably, granule form.

15. Process according to claim 14, wherein an animal feedstuffs additive in the desired powder or granular form is obtained from the fermentation broth, optionally after addition of D-pantothenic acid and/or salts thereof and optionally after addition of organic and inorganic auxiliaries, by
a) drying and compacting, or
b) spray drying, or
c) spray drying and granulation, or
d) spray drying and build-up granulation.
